(19) United States
(12) Patent Application Publication
(10) Pub. No.: US 2005/0261583 A1
(43) Pub. Date: Nov. 24, 2005

(54) Ultrasonic imaging apparatus, ultrasonic image processing method, and ultrasonic image processing program

(75) Inventor: Yoshiaki Satoh, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/131,403

(22) Filed: May 18, 2005

(30) Foreign Application Priority Data
May 20, 2004 (JP) 2004-150025
Dec. 27, 2004 (JP) 2004-377947

(51) Int. Cl. A61B 8/00

(52) U.S. Cl. 600/437

(57) ABSTRACT

An ultrasonic imaging apparatus capable of displaying images in which speckle and structure are expressed in a desired state. The ultrasonic imaging apparatus includes an ultrasonic probe for transmitting ultrasonic waves according to drive signals and receiving ultrasonic echoes generated in an object to obtain detection signals, a drive signal generating unit for generating the drive signals, a signal processing unit for generating original data representing ultrasonic image information on the object by performing signal processing on the detection signals, a speckle/structure separation calculating unit for generating speckle data and structure data based on the original data, a mixed data generating unit for generating mixed data representing information on a mixed image in which a speckle image and a structure image are mixed with each other at a designated ratio, and a DSC for generating image data based on the original data or the mixed data.

### BACKGROUND OF THE INVENTION

1. Field of the Invention

The present invention relates to an ultrasonic imaging apparatus, an ultrasonic image processing method, and an ultrasonic image processing program for generating ultrasonic images based on ultrasonic image signals obtained by transmitting ultrasonic waves and receiving ultrasonic echoes.

2. Description of a Related Art

In medical fields, various imaging technologies have been developed in order to observe the interior of an object to be inspected and make diagnoses. Especially, ultrasonic imaging for obtaining interior information of the object by transmitting and receiving ultrasonic waves enables image observation in real time without exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in the obstetrics, but also gynecology, circulatory system, digestive system, etc.

The ultrasonic imaging is an image generation technology utilizing the nature of ultrasonic waves in which they are reflected at a boundary between regions having different acoustic impedances (e.g., a boundary between structures). Therefore, by transmitting an ultrasonic beam into an object to be inspected such as a human body and receiving ultrasonic echoes generated within the object to obtain reflection points where the ultrasonic echoes are generated or reflection intensity, the outline of a structure (e.g., internal organs, diseased tissues, or the like) existing within the object can be extracted.

By the way, in an ultrasonic image in which an object having a nonuniform structure like a living body is imaged, a pattern having bright parts and/or dark parts are scattered appears. Such pattern is called as a speckle pattern, which is generated by interference between ultrasonic echoes reflected by nonuniform tissues existing within an internal organ, for example. This speckle pattern is a kind of virtual image, and thereby, the demonstrated outline of the structure or the like often becomes unclear.

Japanese Patent Application Publication JP-P2003-61964A discloses an ultrasonic diagnostic apparatus for obtaining tomographic images by applying ultrasonic pulses to an object to be inspected. The ultrasonic diagnostic apparatus includes analysis calculation means for extracting a particular signal by using intensity of echo signals generated from a part of the object or the statistical characteristics of amplitude information, and display means for displaying a result extracted by the analysis calculation means (page 1, FIG. 1). According to the ultrasonic diagnostic apparatus, a small abnormal lesion in a homogeneous tissue structure such as a stage of liver cirrhosis can be observed by using the statistical characteristics of a speckle pattern to perform smoothing images and extracting a small structure.

Further, Ozawa et al., “Endoscopic Ultrasonography Using a Small Probe for Staging of Esophageal Carcinoma”, ENDOSCOPIA DIGESTIVA, 2002, Vol. 14, No. 5, pp. 583-588, describes the study of a diagnosis as to how deep a cancer invades in the layer structure represented in an esophagus tomographic image. When such a diagnosis is performed, an ultrasonic image is desired in which the layer structure appears relatively clearly by removing speckle.

On the other hand, a diagnostic method focused on speckle appearing in an ultrasonic image has been proposed. Japanese Patent Application Publication JP-A-11-125549 discloses an ultrasonic diagnostic apparatus including speckle highlighting means for highlighting a part of echo signals representing a speckle pattern formed by a moving object which diffuses ultrasound echoes reflected by a target to be measured within an object to be inspected, and display means for two-dimensionally displaying the status within the object based on the echo signals and displaying the speckle pattern (pages 1, 3, and 4). According to the ultrasonic diagnostic apparatus, by highlighting the speckle pattern formed by small balls moving with a fluid, information on the flow changing with time can be obtained in spatially accurate positional relationship and real time, and thereby, images representing movement of tissues such as blood stream within blood vessels and heart wall can be formed so that their movements may accurately correspond to spatial positions with respect to a reference image that represents the tissue information in a spatial position.

Further, Kamiyama et al., “Tissue Characterization Using Statistical Information from Ultrasound Echo Signals”, MEDICAL IMAGING TECHNOLOGY, March, 2003, Vol. 21, No. 2, pp. 112-116, discloses general characteristics and statistical characteristics of a speckle pattern appearing in an ultrasonic tomographic image and introduces the study on tissue characteristic diagnoses utilizing such statistical characteristics of the ultrasonic signal.

As described above, in ultrasonic diagnoses, speckle appearing in ultrasonic images is removed or contrary highlighted in accordance with diagnostic purposes. Accordingly, an apparatus that can generate and display various images such as images including only speckle, images including only structure and so on is desired in accordance with various medical diagnoses.

### SUMMARY OF THE INVENTION

The present invention is achieved in view of the above-described problems. A purpose of the present invention is to provide an ultrasonic imaging apparatus capable of displaying images in which speckle and structure are expressed in a desired state, and an ultrasonic image processing method and an ultrasonic image processing program for generating such images.

In order to solve the above-described problems, an ultrasonic imaging apparatus according to one aspect of the present invention includes: ultrasonic transmitting means for transmitting ultrasonic waves according to drive signals; drive signal generating means for generating the drive signals to be provided to the ultrasonic transmitting means; receiving means for receiving ultrasonic echoes generated by reflection of the ultrasonic waves in an object to be inspected so as to obtain detection signals; signal processing means for performing predetermined signal processing on the detection signals to generate original data representing ultrasonic image information on the object; calculating means for generating speckle data representing image information on a speckle and structure data representing image information on a structure based on the original data generated by the signal processing means; and image data generating means for generating image data based on at least one of the original data, the speckle data and the structure data.

Further, an ultrasonic image processing method according to one aspect of the present invention is a method of generating an ultrasonic image based on original data representing ultrasonic image information on an object to be inspected and generated by transmitting ultrasonic waves and receiving ultrasonic echoes generated by reflection of the ultrasonic waves in the object so as to obtain detection signals and performing predetermined signal processing on the detection signals, and the method includes the steps of: (a) generating speckle data representing image information on a speckle and structure data representing image information on a structure based on the original data; and (b) generating image data based on at least one of the original data, the speckle data and the structure data.

Furthermore, an ultrasonic image processing program according to one aspect of the present invention is a program to be used for generating an ultrasonic image based on original data representing ultrasonic image information on an object to be inspected and generated by transmitting ultrasonic waves and receiving ultrasonic echoes generated by reflection of the ultrasonic waves in the object so as to obtain detection signals and performing predetermined signal processing on the detection signals, and the program activates a CPU to execute the procedures of: (a) generating speckle data representing image information on a speckle and structure data representing image information on a structure based on the original data; and (b) generating image data based on at least one of the original data, the speckle data and the structure data.

### BRIEF DESCRIPTION OF THE DRAWINGS

FIG. 1 is a block diagram showing the constitution of an ultrasonic imaging apparatus according to the first embodiment of the present invention;

FIG. 2 shows an original image represented by original data;

FIG. 3 shows the original data on an area as shown by a line A-A′ in FIG. 2;

FIG. 4 is a diagram for explanation of a method of extracting structure data;

FIGS. 5A and 5B show a speckle image and a structure image separated from the original image;

FIG. 6 shows a mixed image of the speckle image and the structure image;

FIG. 7 is a block diagram showing the constitution of an ultrasonic imaging apparatus according to the second embodiment of the present invention;

FIG. 8 shows an example of a composite screen displayed in the ultrasonic imaging apparatus as shown in FIG. 7;

FIG. 9 is a block diagram showing the constitution of an ultrasonic imaging apparatus according to the third embodiment of the present invention;

FIG. 10 shows an example of a composite screen displayed in the ultrasonic imaging apparatus as shown in FIG. 9;

FIG. 11 is a block diagram showing the constitution of an ultrasonic imaging apparatus according to the fourth embodiment of the present invention;

FIG. 12 is a flowchart showing an ultrasonic image processing method according to the fourth embodiment of the present invention;

FIG. 13 is a diagram for explanation of a method of extracting local maximum points of pixels;

FIGS. 14A and 14B are diagrams for explanation of a method of performing four-point interpolation by using square interpolation masks;

FIGS. 15A and 15B are diagrams for explanation of a method of performing four-point interpolation by using flat interpolation masks;

FIG. 16 is a flowchart showing an ultrasonic image processing method according to the fifth embodiment of the present invention;

FIG. 17 is a diagram for explanation of another method of calculating a pixel value of an interpolation point;

FIG. 18 is a block diagram showing the constitution of an ultrasonic imaging apparatus according to the sixth embodiment of the present invention; and

FIG. 19 is a diagram for explanation of frequency band division processing.

### DESCRIPTION OF THE PREFERRED EMBODIMENTS

Hereinafter, embodiments of the present invention will be described in detail by referring to the drawings. The same reference numbers will be assigned to the same component elements and the description thereof will be omitted.

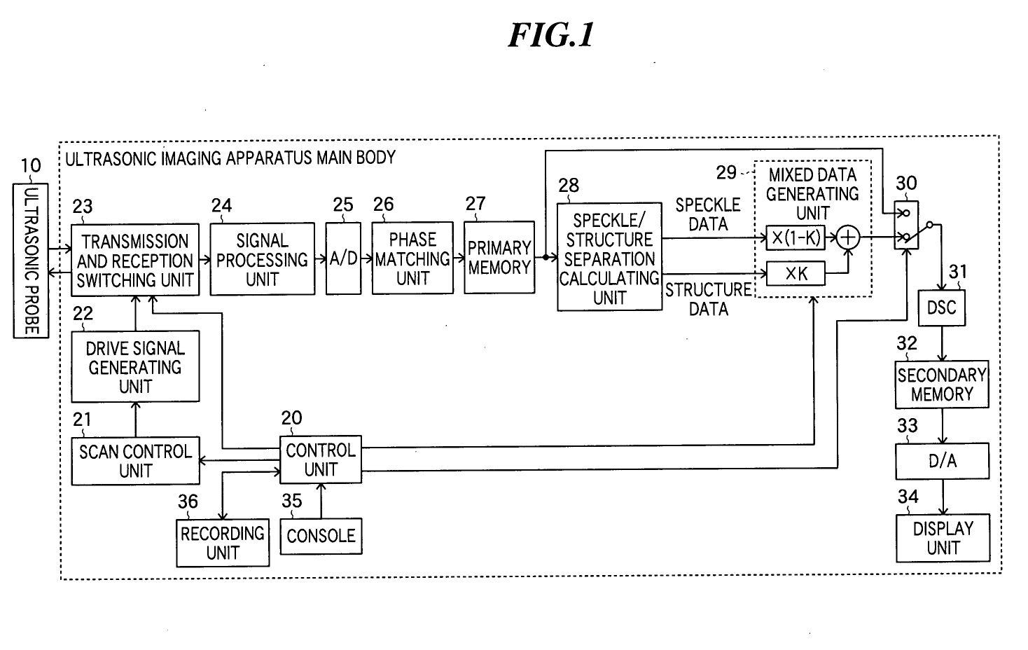

FIG. 1 is a block diagram showing the constitution of an ultrasonic imaging apparatus according to the first embodiment of the present invention. This ultrasonic imaging apparatus includes an ultrasonic probe 10 for transmitting and receiving ultrasonic waves and an ultrasonic imaging apparatus main body for controlling the transmission and reception of ultrasonic waves and generating ultrasonic images based on acquired ultrasonic wave detection signals.

The ultrasonic probe 10 includes an ultrasonic transducer array in which plural ultrasonic transducers are arranged. Each ultrasonic transducer is fabricated by forming electrodes on both ends of a material having a piezoelectric property (piezoelectric material) such as a piezoelectric ceramic represented by PZT (Pb (lead) zirconate titanate) or a polymeric piezoelectric element represented by PVDF (polyvinylidene difluoride). When a voltage is applied to the electrodes of such an ultrasonic transducer by sending pulse electric signals or continuous wave electric signals, the piezoelectric material expands and contracts to generate ultrasonic waves. Accordingly, by electronically controlling plural ultrasonic transducers, pulse or continuous ultrasonic waves are generated from the ultrasonic transducers. Thereby, an ultrasonic beam is formed by combining those ultrasonic waves, and an object to be inspected is electronically scanned. Further, the plural ultrasonic transducers expand and contract by receiving the propagating ultrasonic waves and generate electric signals. These electric signals are outputted as detection signals of the ultrasonic waves. Such an ultrasonic probe 10 is connected to the ultrasonic imaging apparatus main body via a cable.

As the ultrasonic probe 10, a linear array probe in which plural ultrasonic transducers are arranged in one-dimensional manner, a sector probe that can sector-scan within the object, a convex array probe in which plural ultrasonic transducers are arranged on a convex surface, and soon may be used. Further, a two-dimensional array probe in which plural ultrasonic transducers are arranged in a two-dimensional manner may be used. In this case, ultrasonic images on plural different sections can be obtained without mechanically moving the ultrasonic probe.

Alternatively, as the ultrasonic probe 10, a probe within body cavity inserted into the object for performing ultrasonic imaging may be used. As the probe within body cavity, there are known an ultrasonic probe to be used while being inserted into a treatment tool insertion hole of an endoscope, and an ultrasonic endoscope in which an ultrasonic probe is integrated with an endoscope. In the probe within body cavity, ultrasonic imaging is performed in accordance with, for example, a radial scan method. The radials can method includes a mechanical radial scan method of transmitting and receiving ultrasonic waves while rotating the probe and forming ultrasonic images in synchronization with the rotation, and an electronic radial scan method of scanning by electrically controlling plural transducers circularly arranged. According to these scan methods, 360-degree surrounding region of the probe can be displayed at a time. Alternatively, as a probe within body cavity according to another scan method other than the radial scan, one provided with a convex array at the tip end thereof is known. In the case of using the convex array, a wide viewing angle can be obtained.

The ultrasonic imaging apparatus main body includes a control unit 20, a scan control unit 21, a drive signal generating unit 22, a transmission and reception switching unit 23, a signal processing unit 24, an A/D converter 25, a phase matching unit 26, a primary memory 27, a speckle/structure separation calculating unit 28, a mixed data generating unit 29, an image switching unit 30, a DSC (digital scan converter) 31, a secondary memory 32, a D/A converter 33, a display unit 34, a console 35 and a recording unit 36.

The control unit 20 controls each unit within the ultrasonic imaging apparatus.

The scan control unit 21 sets delay times provided to drive signals for driving the plural ultrasonic transducers included in the ultrasonic probe 10 in accordance with directions in which ultrasonic waves are transmitted, under the control of the control unit 20. Further, when a mechanical radial probe is used as the ultrasonic probe 10, the scan control unit 21 controls the motion of a motor for rotating the probe and controls the transmission directions of ultrasonic waves in synchronization with the motion.

The drive signal generating unit 22 includes plural pulsers corresponding to the plural ultrasonic transducers included in the ultrasonic probe 10. Each pulser generates a drive signal with predetermined timing under the control of the scan control unit 21. Thereby, ultrasonic waves are respectively transmitted from the plural ultrasonic transducers with predetermined time difference.

The transmission and reception switching unit 23 switches between input of the drive signals generated in the drive signal generating unit 22 to the ultrasonic probe 10 and load of detection signals in the signal processing unit 24, which will be described later, with predetermined timing in accordance with the control of the control unit 20. Thus, by limiting the time period for loading detection signals, ultrasonic echo signals reflected from a specific depth of the object are detected.

The signal processing unit 24 includes plural channels corresponding to the plural ultrasonic transducers. Each of these channels loads a detection signal outputted from the corresponding ultrasonic transducer with predetermined timing and performs signal processing such as amplification, Nyquist filter processing, and so on.

The A/D converter 25 converts analog signals processed in the signal processing unit 24 into digital signals thereby generates plural pieces of detection data.

The phase matching unit 26 performs reception focus processing by providing delays to detection signals respectively represented by the plural pieces of detection data and adding them. Thereby, detection data (sound ray data) representing a reception beam having a focal point narrowed in the predetermined sound ray direction is generated. By further performing envelope detection with respect to waveforms represented by the sound ray data, image data representing brightness values in plural pixels that form the ultrasonic image is obtained. Hereinafter, this image data is referred to as “original data”.

The primary memory 27 sequentially stores original data generated in the phase matching unit 26. The original data includes information on a structure within the object and information on a speckle pattern.

The speckle/structure separation calculating unit 28 generates a signal representing a speckle pattern, that is, speckle data and a signal representing a structure such as a form of an organ; that is, structure data on the basis of the original data for one frame stored in the primary memory 27.

The mixed data generating unit 29 performs calculation processing based on the speckle data and structure data generated in the speckle/structure separation calculating unit 28 under the control of the control unit 20 to generate mixed data representing image information in which the speckle pattern and structure are mixed at a desired ratio. The mixed data may represent information on only a speckle pattern or information on only a structure.

Under the control of the control unit 20, the image switching unit 30 selects data to be outputted to the DSC 31 from the original data stored in the primary memory 27 and the mixed data generated by the mixed data generating unit 29.

The DSC 31 converts the scan format with respect to the selected data to thereby convert image data representing image information in the sound ray direction within the scan space of the ultrasonic beam into image data for display in physical space. That is, the DSC 31 performs resampling within an image display range and coordinate conversion and interpolation in accordance with the scan method of ultrasonic waves. For example, interpolation processing for generating a linear image is performed on the image data obtained by the linear scan. Further, polar coordinate conversion and interpolation processing are performed on image data obtained by sector scan, convex scan or radial scan.

A STC (sensitivity time control) for correcting distance attenuation may be provided in the previous stage of the DSC 31, or an image processing unit, which performs image processing such as linear gradation processing including gain adjustment and contrast adjustment and non-linear gradation processing including γ correction, may be provided in the subsequent stage of the DSC 31.

Among the above-mentioned components, at least the control unit 20, the speckle/structure separation calculating unit 28, the mixed data generating unit 29, the image switching unit 30 and the DSC 31 may be constructed by a CPU and software (program).

The secondary memory 32 stores image data for display in a format in which raster scan can be performed, for example. Further, the D/A converter 33 converts image data read from the secondary memory 32 into an analog image signal and outputs the analog image signal.

The display unit 34 is a CRT display or an LCD display of a raster scan type, for example, and displays ultrasonic images based on the analog image signal.

The console 35 is used when various instructions and information are inputted to the ultrasonic imaging apparatus main body. The console 35 includes an input device such as a keyboard and touch panel, a pointing device such as a mouse, an adjustment knob, an input button and so on.

The recording unit 36 controls a recording medium for recording programs (software) for activating the CPU included in the ultrasonic imaging apparatus to perform various kinds of processing, information to be used for those processing, etc. As the recording medium, not only the built-in hard disk, an external hard disk, a flexible disk, an MO, an MT, a CD-ROM, a DVD-ROM and so on may be used.

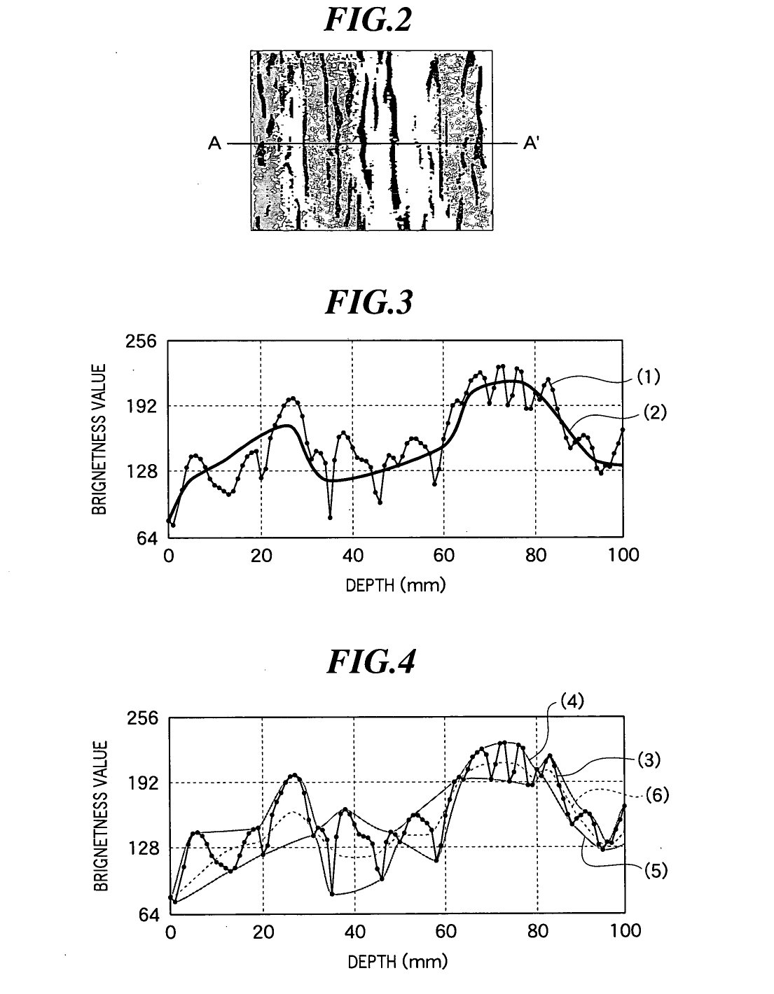

Next, the operation of the ultrasonic imaging apparatus as shown in FIG. 1 will be described by referring to FIGS. 1 to 4. FIGS. 2 to 4 are diagrams for explanation of an ultrasonic image processing method used in the ultrasonic imaging apparatus according to the embodiment.

When an operator starts ultrasonic imaging, the ultrasonic probe 10 as shown in FIG. 1 transmits an ultrasonic beam and scans the object in accordance with the scan method such as linear scan, sector scan, convex scan, radial scan or the like under the control of the control unit 20. This ultrasonic beam is reflected by a reflector existing within the object, and ultrasonic echoes are received by the ultrasonic probe 10. The received ultrasonic echoes are converted into electric signals in the ultrasonic probe 10, and inputted as detection signals to the ultrasonic imaging apparatus main body.

The plural detection signals inputted to the ultrasonic imaging apparatus main body are subjected to predetermined signal processing in the signal processing unit 24, A/D conversion, phase matching and detection processing, and then once stored in the primary memory 27. When the original data for one frame (frame data) is once stored in the primary memory 27, the original data is outputted to the speckle/structure separation calculating unit 28.

FIG. 2 shows an ultrasonic image (original image) represented by the original data stored in the primary memory 27. Further, a curve (1) as shown in FIG. 3 represents original data (brightness values) on a line A-A′ in FIG. 2, and a curve (2) as shown in FIG. 3 represents a signal as to a structure on the line A-A′ in FIG. 2. As shown in FIG. 3, the original data obtained based on the sound ray data includes a signal representing a structure and a signal representing an overlapping speckle pattern. Thus, as shown in FIG. 2, the structure and speckle pattern are mixed in the original image.

Then, the speckle/structure separation calculating unit 28 extracts structure data from the original data as shown in FIG. 3. For this purpose, first, the speckle/structure separation calculating unit 28 obtains a signal representing local maximum points and a signal representing local minimum points in the original data. As shown in FIG. 4, the signal representing local maximum points can be obtained by obtaining derivative values at the respective points in the original data indicated by the curve (3), obtaining points at which the derivative value changes from positive to negative from those derivative values, and further, performing linear interpolation between those points. Similarly, the signals representing local minimum points can be obtained by obtaining points at which the derivative value changes from positive to negative from derivative values at the respective points in the original data indicated by the curve (3), and performing linear interpolation between those points. In FIG. 3, a curve (4) indicates a signal representing the local maximum points and a curve (5) indicates a signal representing the local minimum points.

Here, if it was determined whether or not a certain point in the original data is adopted as a local maximum/minimum point simply based on the derivative values in the original data, then the case might occur where the local maximum/minimum point caused by the speckle and the local maximum/minimum point caused by the structure are mixed. Accordingly, as a determination condition as to whether or not a certain point in the original data is adopted as a local maximum/minimum point, the following condition is desirably added. That is, the distance between the certain point and the local maximum/minimum point, which was extracted immediately before, is calculated, and the certain point is not adopted as a local maximum/minimum point if the distance is longer than the wavelength of the transmitted ultrasonic wave.

Next, the speckle/structure separation calculating unit 28 obtains a signal representing average values of the signal representing the local maximum points and the signal representing the local minimum points. A curve (6) in FIG. 4 indicates the signal representing the average values. The signal representing the average values form the structure data representing the ultrasonic image of the structure (structure image) within the imaging area. Alternatively, the signal representing the local maximum points or the signal representing the local minimum points may be used as the structure data.

Furthermore, the speckle/structure separation calculating unit 28 calculates speckle data by subtracting the values represented by the structure data from the values represented by the original data. At the time, an offset value may be added to the difference values thereof according to need. As the offset value, a fixed value that has been set in advance in the ultrasonic imaging apparatus may be used, or a value inputted by an operator may be used.

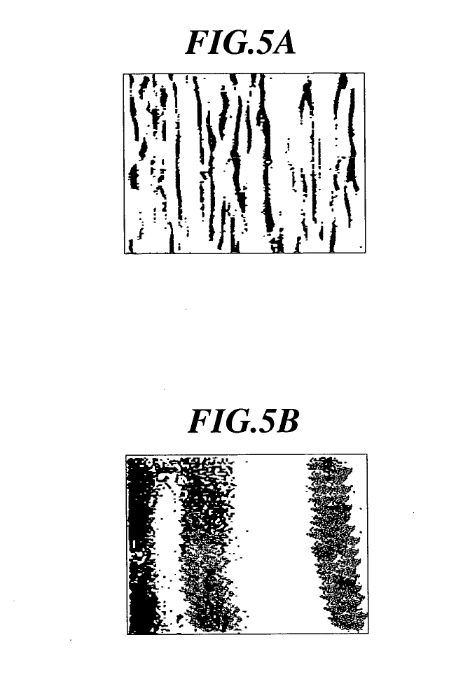

By performing such calculation processing with respect to frame data for one frame, a speckle image as shown in FIG. 5A and a structure image as shown in FIG. 5B separated from each other can be obtained.

The following is the reason why the local maximum points and the local minimum points in the original data are used in the embodiment rather than a general filter processing when the signal representing the structure is obtained. That is, the size of the speckle pattern expressed in the imaging area (speckle size) differs depending on the depth of the imaging area. Accordingly, when the filter processing is uniformly performed on the original data, the case occurs where the speckle can not be removed completely, or contrary, the signal representing the structure is removed.

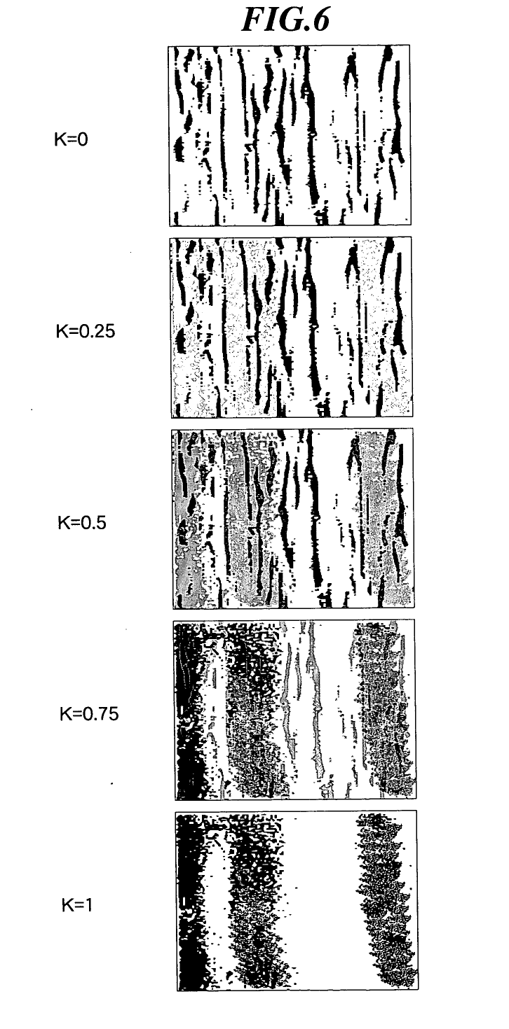

Referring to FIG. 1 again, the mixed data generating unit 29 calculates values of mixed data by using the following equation based on the values represented by the speckle data and the values represented by the structure data generated in the speckle/structure separation calculating unit 28.

$$( mixed data value ) = ( structure data value ) \times K + ( speckle data value ) \times ( 1 - K )$$

Where K represents mixing ratio between the structure image and the speckle image, and a desired value within a range 0≦K≦1 is inputted by the operator using the console 35. Thereby, as shown in FIG. 6, the mixed image in which the structure and speckle pattern are mixed at the desired ratio can be obtained. If K=0, the mixed image is an image including only a speckle pattern (i.e., speckle image), and, if K=1, the mixed image is an image including only a structure (i.e., structure image).

Furthermore, the operator selects one of the original image and the mixed image to be displayed on the display unit 34, and inputs an instruction by using the console 35. In response to this, the image switching unit 30 changes over the switch 30 under the control of the control unit 20. Thereby, one of the original data stored in the primary memory 27 and the mixed data generated in the mixed data generating unit 29 is outputted to the DSC 31. The selected image data is subjected to the predetermined processing in the DSC 31, converted into an analog signal by the D/A converter 33, and displayed on the display unit 34. Thereby, the selected image is displayed on the display unit 34.

As described above, according to the embodiment, since the mixing ratio K as a variable value is inputted by the operator, an image in which the structure and speckle pattern are mixed at the desired ratio (including an image of only speckle or only structure) can be generated. Further, such mixed image and the original image can be displayed on the screen in a form desired by the operator. Therefore, appropriate images are displayed on the screen in accordance with diagnostic purposes, and thereby, the diagnoses by doctors are facilitated and the quality of diagnoses can be improved.

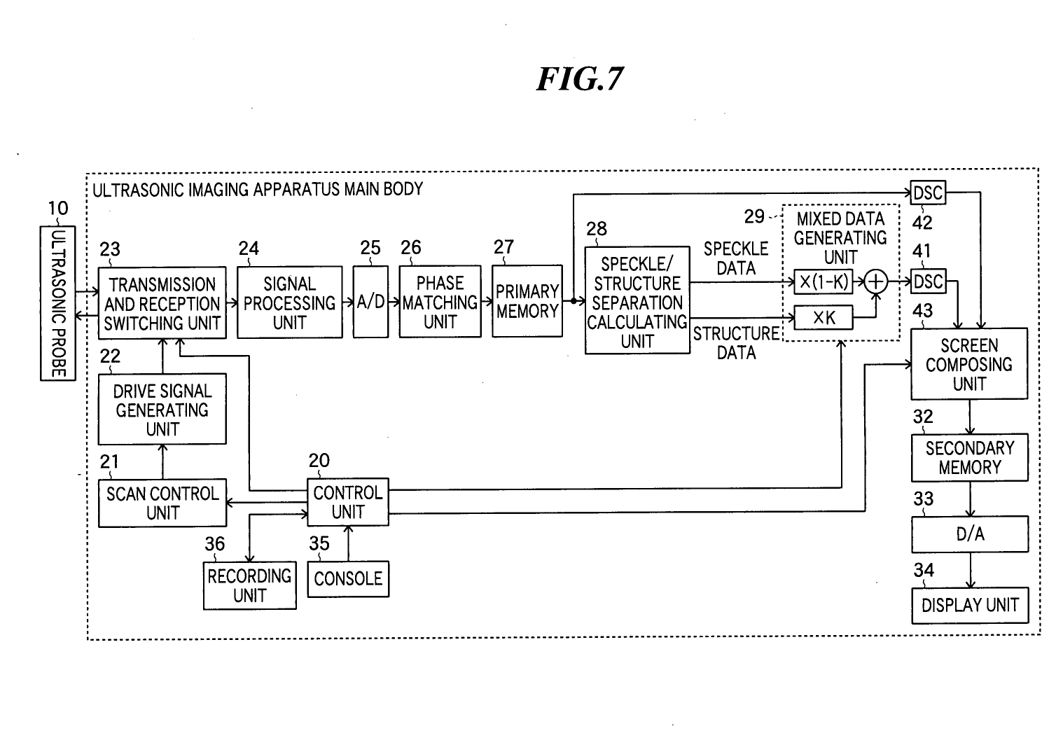

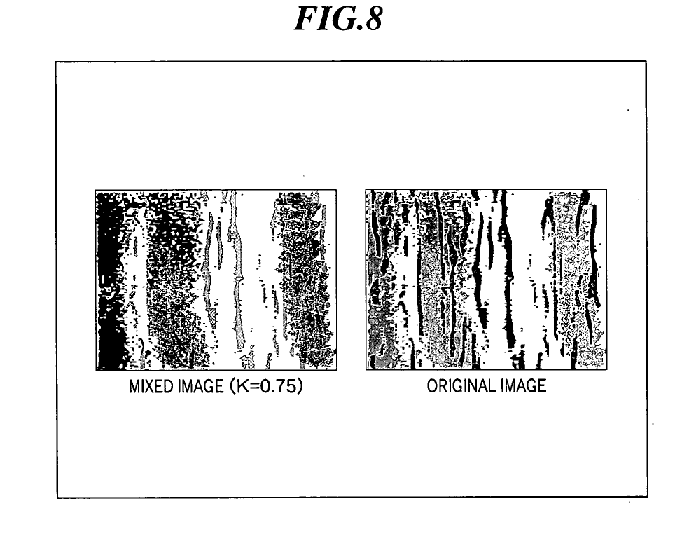

Next, an ultrasonic imaging apparatus according to the second embodiment of the present invention will be described by referring to FIGS. 7 and 8.

The ultrasonic imaging apparatus as shown in FIG. 7 includes a first DSC 41 and a second DSC 42 in place of the DSC 31 as shown in FIG. 1, and further includes a screen composing unit 43. Other constitution is the same as in the ultrasonic imaging apparatus shown in FIG. 1. The first DSC 41, the second DSC 42 and the screen composing unit 43 also may be constructed by the CPU and software (program).

The DSC 41 performs conversion of scan format with respect to the mixed data generated by the mixed data generating unit 29. On the other hand, the DSC 42 performs conversion of scan format with respect to the original data stored in the primary memory 27. Further, the screen composing unit 43 creates a screen to be displayed on the display unit 34 based on the mixed data and the original data outputted from the DSC 41 and the DSC 42, respectively, under the control of the control unit 20.

The screen to be created by the screen composing unit 43 is set based on the instruction inputted by the operator using the console 35. According to the instruction inputted by the operator, the screen composing unit 43 creates a screen in which only the mixed image is displayed, a screen in which the original image is displayed, or a screen in which the mixed image and the original image are displayed side by side (FIG. 8). At that time, the operator can adjust the mixing ratio K to be used in the mixed data generating unit 29 such that the image including only the speckle pattern or the image including only the structure and the original image can be displayed side by side.

As described above, according to the embodiment, one of the original image and the mixed image, or both of them can be displayed according to operator's preference. Especially, in the latter case, since the original image and the mixed image can be compared and referred to in one screen, the efficiency of the diagnoses by doctors can be improved.

Here, in the embodiment, the scan format conversions are performed with respect to mixed data and original data in parallel by providing the two DSC. However, the scan format conversions may be performed with respect to those image data by one DSC in a time-sharing manner, and thus obtained two kinds of image data may be outputted to the screen composing unit 43.

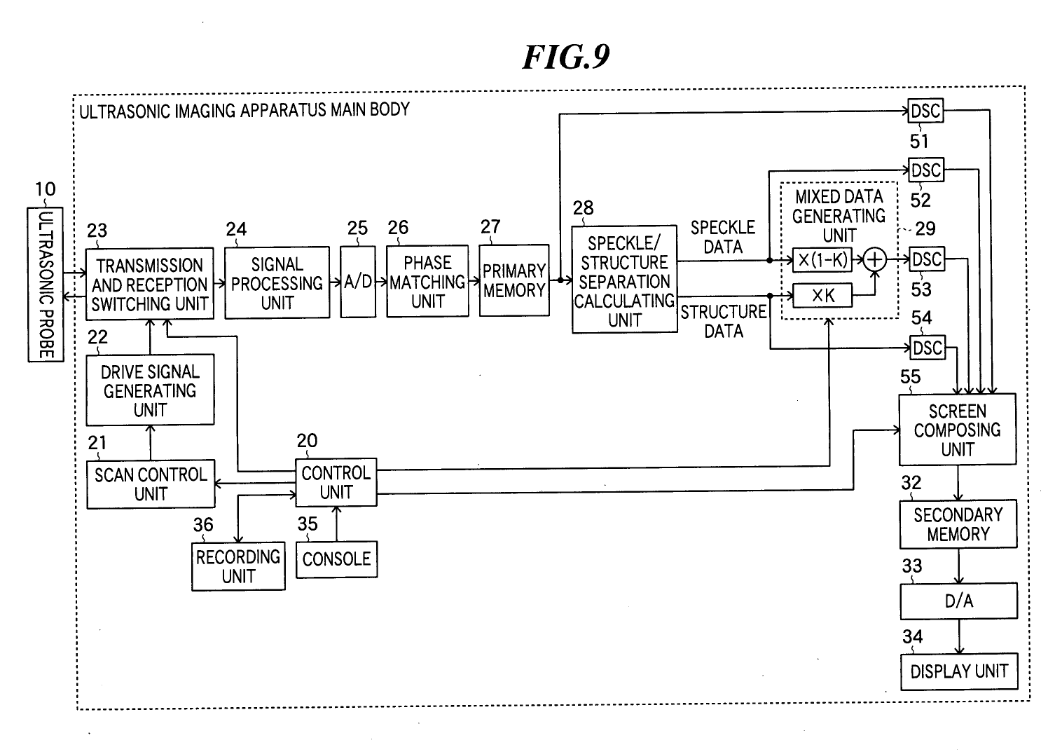

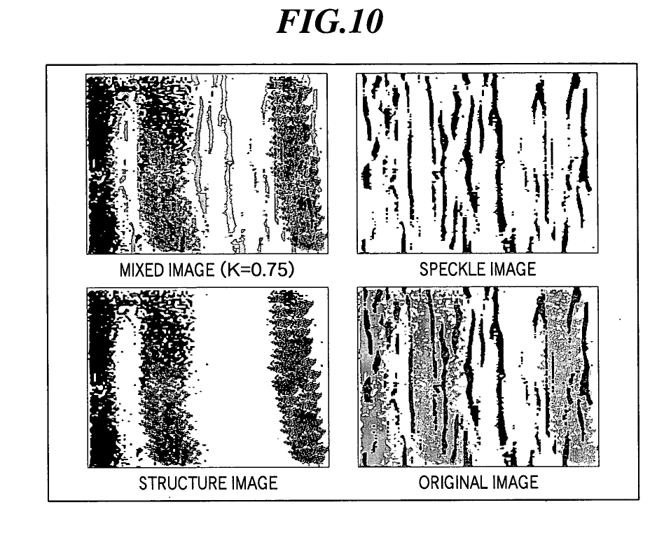

Next, an ultrasonic imaging apparatus according to the third embodiment of the present invention will be described by referring to FIGS. 9 and 10.

The ultrasonic imaging apparatus as shown in FIG. 9 has first to fourth DSCs 51 to 54 in place of the DSC 31 as shown in FIG. 1, and further includes a screen composing unit 55. Other constitution is the same as in the ultrasonic imaging apparatus as shown in FIG. 1. The first to fourth DSCs 51 to 54 and the screen composing unit 55 also may be constructed by the CPU and software (program).

The DSC 51 performs conversion of scan form at with respect to the original data stored in the primary memory 27. Further, the DSC 52 performs conversion of scan format with respect to the speckle data generated in the speckle/structure separation calculating unit 28. Further, the DSC 53 performs conversion of scan format with respect to the mixed data generated by the mixed data generating unit 29. Further, the DSC 53 performs conversion of scan format with respect to the structure data generated in the speckle/structure separation calculating unit 28. The screen composing unit 55 creates a screen to be displayed on the display unit 34 based on the image data outputted from the DSCs 51 to 54 under the control of the control unit 20.

The screen created by the screen composing unit 55 is set based on the instruction inputted by the operator using the console 35. That is, the screen composing unit 55 creates a screen in which one image selected from the original image, the speckle image, the mixed image and the structure image is displayed, or creates a composite screen in which two to four images are displayed side by side (FIG. 10). Thereby, the operator can make a medical diagnosis by using an ultrasonic image displayed in the desired form.

By the way, in the embodiment, instead of providing the four DSCs, the scan format conversions may be performed with respect to the original data, the speckle data, the mixed data and the structure data by one DSC in a time-sharing manner, and thus obtained four kinds of image data may be outputted to the screen composing unit 55.

In the above-described first to third embodiments of the present invention, in order to separate the speckle data and the structure data, the local maximum points and/or the local minimum points in the original data are used. However, other methods may be used. For example, the structure data may be obtained by extracting the speckle data by using the statistical characteristics of intensity or amplitude information of echo signals (see page 4 of the publication JP-P2003-61964A, or page 114 of Kamiyama et al., “Tissue Characterization Using Statistical Information from Ultrasound Echo Signals”), and subtracting the values represented by the speckle data from the values represented by the original data to obtain the structure data.

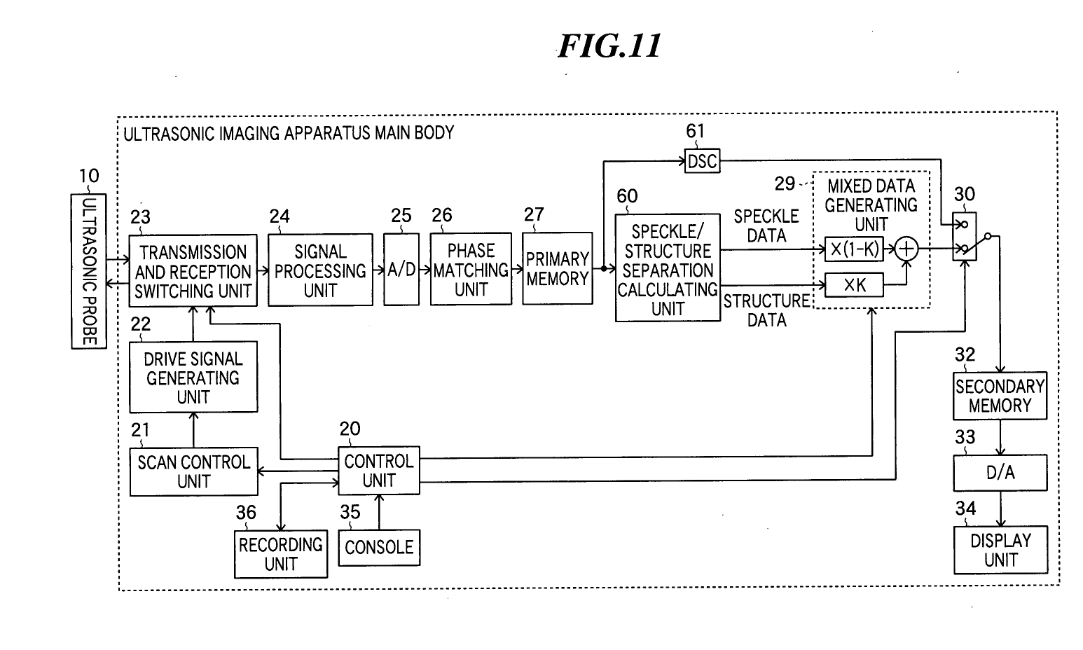

Next, an ultrasonic imaging apparatus according to the fourth embodiment of the present invention will be described by referring to FIGS. 11 to 15B.

FIG. 11 is a block diagram showing the constitution of an ultrasonic imaging apparatus according to the embodiment. This ultrasonic imaging apparatus has a speckle/structure separation calculating unit 60 in place of the speckle/structure separation calculating unit 28 as shown in FIG. 1 and a DSC 61 in place of the DSC 31 shown in FIG. 1. The speckle/structure separation calculating unit 60 and the DSC 61 also may be constructed by the CPU and software (program).

The speckle/structure separation calculating unit 60 generates speckle data and structure data, which have been subjected to interpolation processing and scan conversion, based on the original data for one frame stored in the primary memory 27. Further, the DSC 61 performs conversion of scan format with respect to the original data stored in the primary memory 27.

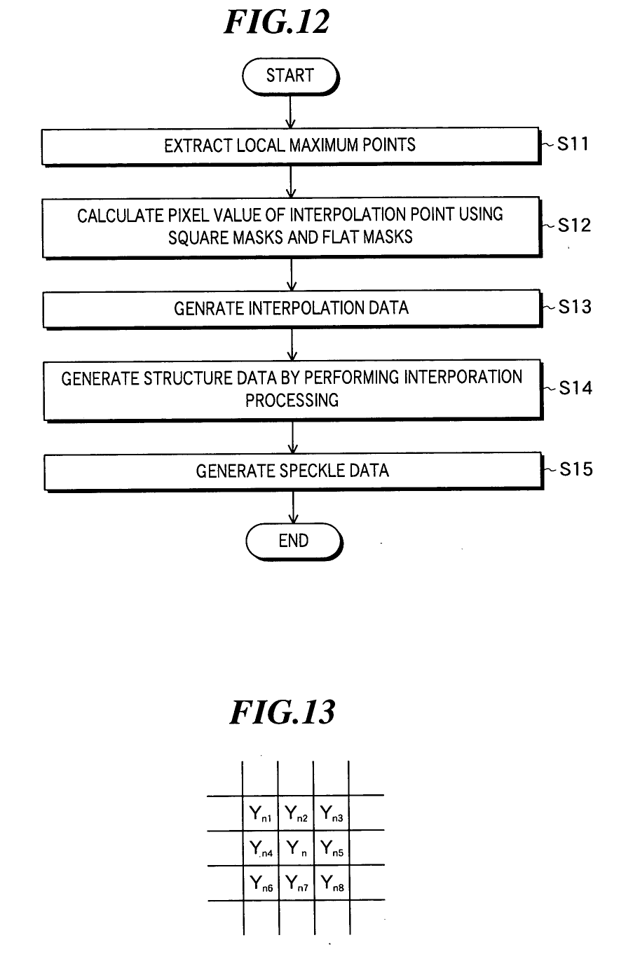

FIG. 12 is a flowchart showing an ultrasonic image processing method to be used in the ultrasonic imaging apparatus according to the embodiment. The ultrasonic image processing method according to the embodiment is performed on the original data acquired by performing linear scan and characterized by using two-dimensional mask processing at that time.

At step S11 in FIG. 12, the speckle/structure separation calculating unit 60 as shown in FIG. 11 extracts local maximum points from the original image based on the original data stored in the primary memory 27. The local maximum points can be extracted by, for example, the following method. As shown in FIG. 13, attention is focused on a certain pixel Yₙ, and the pixel value (brightness value) D(Yₙ) of the pixel Yₙ and pixel values (Yₙ₁) to D(Yₙ₈) of pixels Yₙ₁ to Yₙ₈ located in the periphery thereof are compared. Then, if the pixel value D(Yₙ) is larger than the pixel values of the surrounding pixels, that is, all of the relational expressions D(Yₙ)>(Yₙ₁), D(Yₙ)>(Yₙ₂), D(Yₙ)>(Yₙ₃), D(Yₙ)>(Yₙ₄), D(Yₙ)>(Yₙ₅), D(Yₙ)>(Yₙ₆), D(Yₙ)>(Yₙ₇), and D(Yₙ)>(Yₙ₈) are satisfied, Yₙ is judged as a local maximum point.

Then, at step S12 in FIG. 12, the speckle/structure separation calculating unit 60 calculates a pixel value of an interpolation point by four-point interpolation using square interpolation masks or flat interpolation masks in order to interpolate the extracted local maximum points.

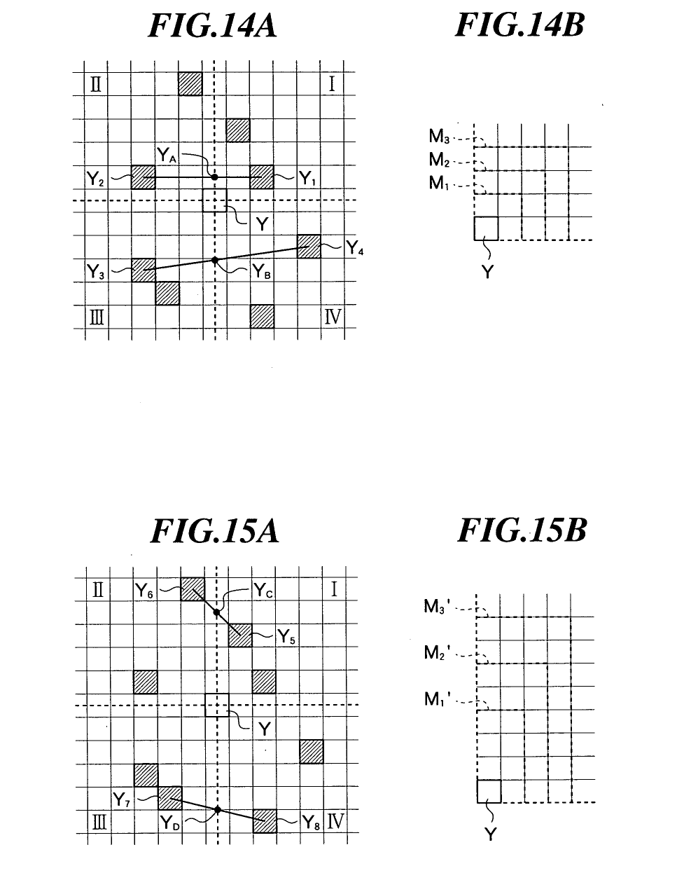

FIGS. 14A and 14B are diagrams for explanation of a method of performing the four-point interpolation using square interpolation masks, and shaded areas show local maximum points extracted at step S11. As shown in FIG. 14A, in order to obtain four points to be used for calculating a pixel value of the interpolation point Y, first, with the interpolation point Y as a center, surrounding pixels are divided into four quadrants. Then, by using the square interpolation masks M₁, M₂ . . . as shown in FIG. 14B sequentially in ascending order of mask size, the local maximum points near the interpolation point Y are explored in the first quadrant to the fourth quadrant, respectively. The pixels Y₁ to Y₄ as shown in FIG. 14A show the local maximum points respectively explored in the first quadrant to the fourth quadrant.

Then, the pixel value D(Y) of the interpolation point Y is calculated based on the positions of the explored pixels Y₁ to Y₄ and the pixel values D(Y₁) to D(Y₄). For this purpose, a pixel value D(Y_{A}) at a point Y_{A} on one axis including the pixel Y is calculated by the weighed average method using the pixel value D(Y₁) of the pixel Y₁ and the pixel value D(Y₂) of the pixel Y₂, and the pixel value D(Y_{B}) at a point Y_{B} on another axis including the pixel Y is calculated by using the pixel value D(Y₃) of the pixel Y₃ and the pixel value D(Y₄) of the pixel Y₄. Further, the pixel value D(Y) of the interpolation point Y is calculated by the weighed average method using the positions of the points Y_{A} and Y_{B} and the pixel values D(Y_{A}) and D(Y_{B}).

FIGS. 15A and 15B are diagrams for explanation of a method of performing four-point interpolation using flat interpolation masks. In this embodiment, non-square masks extending in the vertical direction in the drawing (i.e., depth direction in the object) are used. As shown in FIG. 15, in order to obtain four points to be used for calculating the pixel value of the interpolation point Y, first, with the interpolation point Y as a center, surrounding pixels are divided into four quadrants. Then, by using the flat interpolation masks M₁′, M₂′ . . . as shown in FIG. 15B sequentially in ascending order of mask size, the local maximum points near the interpolation point Y are explored in the first quadrant to the fourth quadrant, respectively. In FIG. 15A, the pixels Y₅ to Y₈ show the local maximum points respectively in the first quadrant to the fourth quadrant.

Then, the pixel value D(Y)′ of the interpolation point Y is calculated based on the positions of the explored pixels Y₅ to Y₈ of the explored local maximum points and the pixel values D(Y₅) to D(Y₈). The method of calculating the pixel value D(Y)′ is the same as that for the square interpolation masks.

Then, at step S13 in FIG. 12, interpolation data is generated based on the pixel value D(Y) calculated by using the square interpolation masks and the pixel value D(Y)′ calculated by using the flat interpolation masks.

Here, the image interpolation processed by using the square interpolation masks is good in continuousness in lateral lines, however, not very good in continuousness in diagonal lines. On the other hand, the image interpolation processed by using the flat interpolation masks is good in continuousness in diagonal lines, however, not very good in continuousness in lateral lines. Accordingly, in the embodiment, the interpolation data is generated by comparing the pixel value D(Y) calculated by using the square interpolation masks and the pixel value D(Y)′ calculated by using the flat interpolation masks, and adopting the value of the larger pixel value as the pixel value in the interpolation point Y.

Then, at step S14, by using the interpolation data generated at step S13, interpolation processing is performed with respect to data at local maximum points extracted at step S11. Thus generated image data forms structure image data representing an ultrasonic image of the structure (structure image) within the imaging area.

Next, at step S15, the speckle data representing the speckle image is generated by subtracting the values represented by the structure data generated at step S14 from the values represented by the original data.

According to the embodiment, since the interpolation processing is performed based on the pixel values obtained by using the square interpolation masks and the flat interpolation masks, the structure image good in continuousness in lateral lines and diagonal lines can be obtained. Therefore, by using such a structure image, a speckle image good in separation from the structure can be obtained.

As a modified example of the ultrasonic image processing method according to the embodiment, interpolation data may be generated by using one of the square interpolation masks and the flat interpolation masks at steps S12 and S13 as shown in FIG. 12. In the case of using the square interpolation masks, a structure image good in continuousness in lateral lines can be acquired, and in the case of using the flat interpolation masks, a structure image good in continuousness in diagonal lines can be acquired. Alternatively, when the interpolation data is generated at step S13, one of the pixel values obtained by using the square interpolation masks, the pixel values obtained by using the flat interpolation masks, and the pixel values selected by comparing those pixel values may be selected. Thereby, a structure image desired by the operator can be acquired.

Next, an ultrasonic image processing method according to the fifth embodiment of the present invention will be described by referring to FIGS. 11 to 14B and 16. The ultrasonic image processing method according to the embodiment is performed on the original data acquired by performing sector scan, convex scan, or radial scan and characterized by using two-dimensional mask processing at that time, and can be used in the speckle/structure separation calculating unit 60 as shown in FIG. 11.

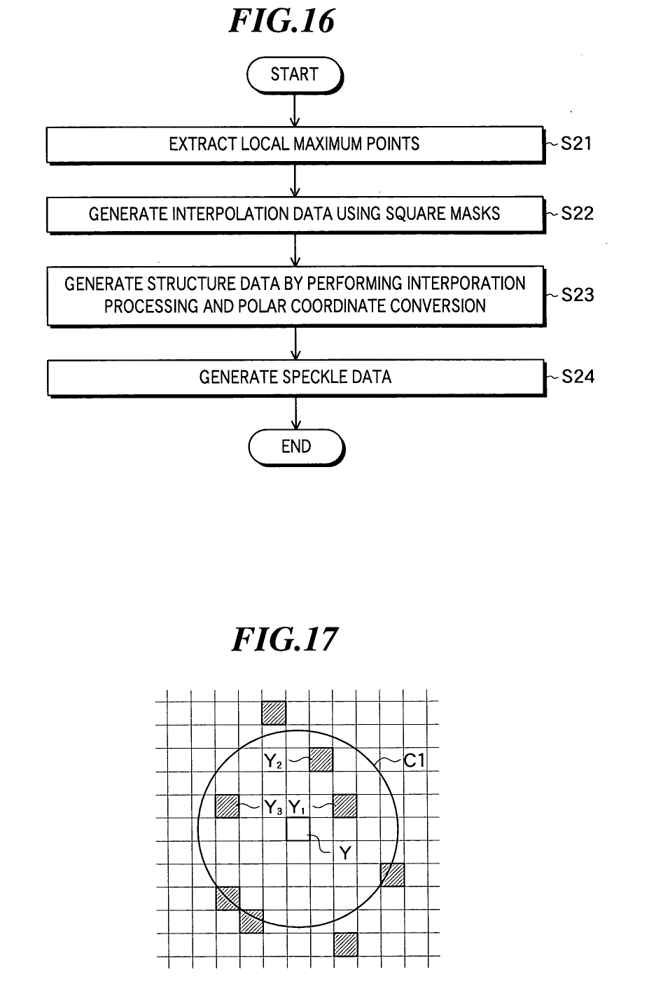

FIG. 16 is a flowchart showing the ultrasonic image processing method according to the embodiment.

At step S21 in FIG. 16, the speckle/structure separation calculating unit 60 as shown in FIG. 11 extracts local maximum points from the original image based on the original data stored in the primary memory 27. Then, at step S22, the speckle/structure separation calculating unit 60 generates interpolation data by calculating the pixel value of the interpolation point for interpolation of local maximum points by four-point interpolation using square interpolation masks. Note that the extraction processing of the local maximum points at step S21 and the calculation processing of the pixel value at S22 are the same as described at steps S11 and S12 in FIG. 12 by referring to FIGS. 13 to 14B.

Then, at step S23, interpolation processing is performed with respect to data at the local maximum points extracted at step S21 by using the interpolation data generated at step S22, and further, polar coordinate conversion processing (scan conversion processing) in accordance with the scan method of ultrasonic wave is performed. Thereby, image data representing a sector image, convex image, or radial image is generated. Such image data forms structure data representing structure image in the imaging area.

Then, at step S24, the speckle data representing the speckle image is generated by subtracting the values represented by the structure data from the values represented by the original data.

According to the embodiment, since the interpolation processing and the polar coordinate conversion processing are performed based on the pixel value calculated by using the square interpolation masks good in continuousness in lateral lines, a structure image good in continuousness can be obtained. Therefore, by using such a structure image, a speckle image good in separation from the structure can be obtained.

In the fourth and fifth embodiments described above, the pixel value of the interpolation point is calculated by performing the four-point interpolation using local maximum points. However, the pixel value may be calculated in accordance with other methods. For example, as shown in FIG. 17, pixel values of the local maximum points Y₁, Y₂, Y₃ . . . included within a predetermined range from the interpolation point Y (e.g., inside of a circle Cl around the interpolation point Y as a center). In this case, the pixel value D(Y) of the interpolation point Y can be calculated by the following equation (1). In the equation (1), d₁, d₂, and d₃ represent the distances between the interpolation point Y and the local maximum points Y₁, Y₂, and Y₃, respectively.

$$D ( Y ) = ( 1 / {d}_{1} ) ( 1 / {d}_{1} + 1 / {d}_{2} + 1 / {d}_{3} ) \times {Y}_{1} + ( 1 / {d}_{2} ) ( 1 / {d}_{1} + 1 / {d}_{2} + 1 / {d}_{3} ) \times {Y}_{2} + ( 1 / {d}_{3} ) ( 1 / {d}_{1} + 1 / {d}_{2} + 1 / {d}_{3} ) \times {Y}_{3} \qquad ( 1 )$$

According to the method, the time for exploring the pixels of the local maximum points to be used when the pixel value of the pixel Y is calculated can be shortened.

Alternatively, in place of the equation (1), the pixel value of the pixel Y may be calculated using the following equation (2) or (3).

$$\begin{matrix}\begin{matrix}D ( Y ) = ( 1 / {d}_{1}^{2} ) ( 1 / {d}_{1}^{2} + 1 / {d}_{2}^{2} + 1 / {d}_{3}^{2} ) \times {Y}_{1} \\ = ( 1 / {d}_{2}^{2} ) ( 1 / {d}_{1}^{2} + 1 / {d}_{2}^{2} + 1 / {d}_{3}^{2} ) \times {Y}_{2} \\ = ( 1 / {d}_{3}^{2} ) ( 1 / {d}_{1}^{2} + 1 / {d}_{2}^{2} + 1 / {d}_{3}^{2} ) \times {Y}_{3}\end{matrix} & ( 2 ) \\ \begin{matrix}D ( Y ) = ( 1 / {d}_{1}^{3} ) ( 1 / {d}_{1}^{3} + 1 / {d}_{2}^{3} + 1 / {d}_{3}^{3} ) \times {Y}_{1} \\ = ( 1 / {d}_{2}^{3} ) ( 1 / {d}_{1}^{3} + 1 / {d}_{2}^{3} + 1 / {d}_{3}^{3} ) \times {Y}_{2} \\ = ( 1 / {d}_{3}^{3} ) ( 1 / {d}_{1}^{3} + 1 / {d}_{2}^{3} + 1 / {d}_{3}^{3} ) \times {Y}_{3}\end{matrix} & ( 3 )\end{matrix}$$

In the case of using the equation (2) or (3), the local maximum points near the pixel Y exert a larger influence compared to the case of using the equation (1).

Further, in the fourth and fifth embodiments of the present invention, the structure image data is generated by using the local maximum points in the original image. However, in place of the local maximum points, local minimum points or average points of the local maximum points and the local minimum points may be used.

Furthermore, in the fourth and fifth embodiments of the present invention, a screen composing unit that creates a screen for display based on the original image data, the structure data, the speckle data and the mixed data may be provided similarly to that in the second or third embodiment of the present invention.

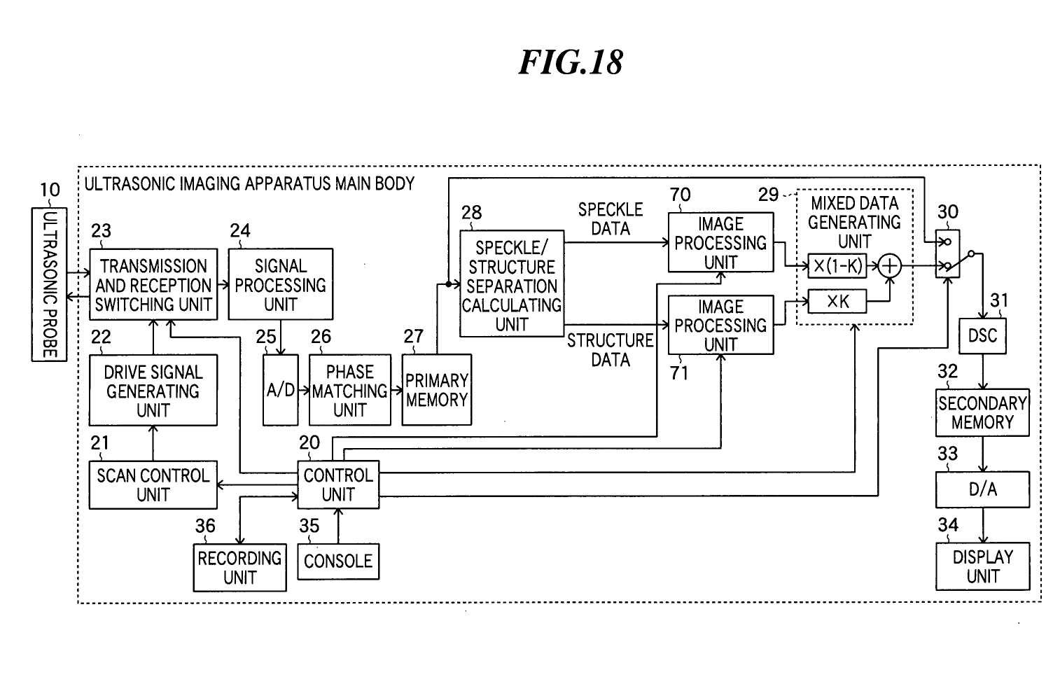

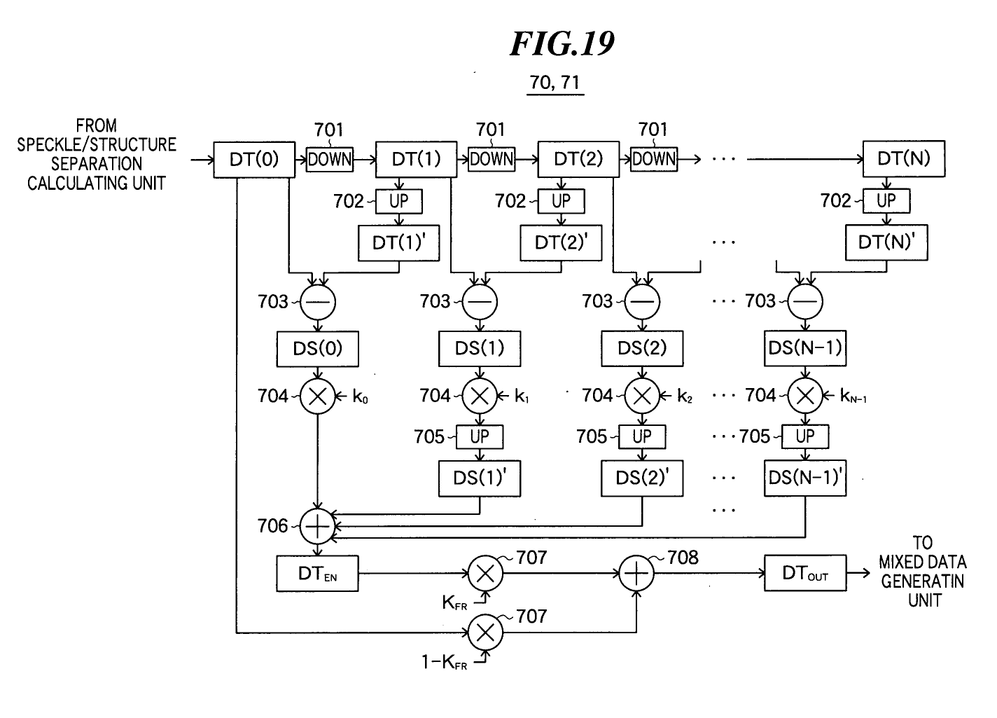

Next, an ultrasonic imaging apparatus according to the sixth embodiment of the present invention will be described by referring to FIGS. 18 and 19.

FIG. 18 is a block diagram showing the constitution of the ultrasonic imaging apparatus according to the embodiment. The ultrasonic imaging apparatus as shown in FIG. 18 further includes image processing units 70 and 71 in comparison with the ultrasonic imaging apparatus as shown in FIG. 1. Other constitution is the same as in the ultrasonic imaging apparatus as shown in FIG. 1.

The image processing unit 70 performs frequency band division processing on the speckle data generated in the speckle/structure separation calculating unit 28. Further, the image processing unit 71 performs frequency band division processing on the structure data generated in the speckle/structure separation calculating unit 28. The frequency band division processing refers to image processing of dividing an ultrasonic image into plural frequency bands and enhancing a desired frequency component.

The frequency band division processing performed in each of the image processing units 70 and 71 will be described in detail by referring to FIG. 19. FIG. 19 is a diagram for explanation of the frequency band division processing.

As shown in FIG. 19, when speckle data or structure data (image data) DT(0) generated in the speckle/structure separation calculating unit 28 is inputted, in a down-sampling unit 701, the image data DT(0) is thinned out and filter processing such as Nyquist filter processing is performed on the thinned out data. By repeating such processing, down-sampling data DT(1), DT(2), . . . , DT(N) having low spatial frequency components are sequentially generated.

Then, in an up-sampling unit 702, “0” value data is inserted into the “n”th down-sampling data DT(n)(n=1 to N) and filter processing such as smoothing filter processing is performed. Thereby, up-sampling data DT(n)′ having the same size as that of the adjacent (n−1)th data is obtained.

Next, in a subtracting unit 703, subtraction processing is performed between the (n−1)th down-sampling data DT(n−1) and the adjacent “n”th up-sampling data DT(n)′. Thereby, subtraction data DS(0) to DS(N−1) are obtained. These subtraction data DS(0) to DS (N−1) are data groups including frequency components that are formed by dividing spatial frequency components f₀ to f_{N} included in the image data DT(0) into N frequency bands, respectively. For example, the subtraction data DS(n) (n=0 to N−1) includes frequency components fₙ to fₙ₊₁.

Then, in a multiplying unit 704, subtraction data DS(0), DS(1), . . . , DS(N−1) are multiplied by weighting factors k₀, k₁, . . . , k_{N-1}, respectively. Furthermore, the data DS(n)′ (n=1 to N−1) multiplied by the weighting factors are up-sampled in the up-sampling unit 705 so as to have the same data size as that of the original image data DT(0).

Thus equally sized data DS(0) and DS(1)′, DS(2)′, . . . , DS(N−1)′ are added to each others in an adding unit 706. Thereby, data DT_{EN} weighted with respect to each spatial frequency band is generated. Furthermore, the weighted data DT_{EN} and the original image data DT(0) are multiplied by predetermined weighting factors K_{FR} and (1−K_{FR}), respectively, in the multiplying unit 707, and added to each other in the adding unit 708. Thus, image data DT_{OUT} that has been subjected to the frequency enhancement processing is generated, and outputted to the mixed data generating unit 29.

The weighting factors k₀ to k_{N-1} to be used in the multiplying unit 704 are set in accordance with the characteristics of the image data to be processed. The weighting factors k₀ to k_{N-1} may be stored in the recording unit 36 as shown in FIG. 18 in advance in association with parameters such as the ultrasonic frequency, the depth of the object or observation part. Alternatively, the operator may input arbitrary values. In the former case, the weighting factors suitable for those parameters are set, and in the latter case, operator-desired frequency enhancement effect can be obtained.

In the case where such frequency band division processing is performed on the speckle data, speckle having a large size is reduced by making the vicinity of the weighting factors k₄ and k₅ smaller to suppress the smaller frequency components. Thereby, an image, in which a tissue part is easily viewable, can be obtained in the synthesized image. On the other hand, in the case such frequency band division processing is performed on the structure data, the effect of making edges of the structure clearer can be obtained by making the vicinity of the weighting factors k₀ and k₁ larger to enhance the larger frequency components.

Further, in the embodiment, in the image processing units 70 and 71 as shown in FIG. 18, various kinds of image processing may be performed in spite of the frequency band division processing. Specifically, smoothing filter processing, Laplacian filter processing, etc. may be performed.

Furthermore, the image processing units 70 and 71 as shown in FIG. 18 and described in the embodiment may be applied to the ultrasonic imaging apparatuses according to the second to fourth embodiments of the present invention as shown in FIGS. 7, 9, and 11.

As described above, according to the present invention, since the speckle data and the structure data are separated from the acquired original data and an image is generated based on the data selected from those data or by mixing speckle and structure at a desired ratio, the original image, the image including only speckle or structure, or the image in which they are mixed at a desired ratio can be displayed in accordance with the selection by the operator. Thus, by using appropriate images, medical diagnoses by doctors are facilitated and diagnosis efficiency can be improved, and therefore, the quality of diagnoses can be improved.

1. An ultrasonic imaging apparatus comprising:
ultrasonic transmitting means for transmitting ultrasonic waves according to drive signals;
drive signal generating means for generating the drive signals to be provided to said ultrasonic transmitting means;
receiving means for receiving ultrasonic echoes generated by reflection of the ultrasonic waves in an object to be inspected so as to obtain detection signals;
signal processing means for performing predetermined signal processing on the detection signals obtained by said receiving means to generate original data representing ultrasonic image information on the object;
calculating means for generating speckle data, representing image information on a speckle and structure data representing image information on a structure based on the original data generated by said signal processing means; and
image data generating means for generating image data based on at least one of the original data, the speckle data and the structure data.

2. An ultrasonic imaging apparatus according to claim 1, further comprising:
mixed data generating means for generating mixed data representing information on a mixed image in which a speckle image and a structure image are mixed with each other at a designated ratio based on the speckle data and the structure data;
wherein said image data generating means generates image data based on at least one of the original data, the speckle data, the structure data and the mixed data.

3. An ultrasonic imaging apparatus comprising:
ultrasonic transmitting means for transmitting ultrasonic waves according to drive signals;
drive signal generating means for generating the drive signals to be provided to said ultrasonic transmitting means;
receiving means for receiving ultrasonic echoes generated by reflection of ultrasonic waves in an object to be inspected so as to obtain detection signals;
signal processing means for performing predetermined signal processing on the detection signals obtained by said receiving means to generate original data representing ultrasonic image information on the object;
calculating means for generating speckle data representing image information on a speckle and structure data representing image information on a structure based on the original data generated by said signal processing means;
mixed data generating means for generating mixed data representing information on a mixed image in which a speckle image and a structure image are mixed with each other at a designated ratio based on the speckle data and the structure data; and
image data generating means for generating image data based on at least one of the original data and the mixed data.

4. An ultrasonic imaging apparatus according to claim 2, wherein the ratio at which the speckle image and the structure image are mixed with each other can be designated in a range from 1:0 to 0:1.

5. An ultrasonic imaging apparatus according to claim 2, wherein said image data generating means generates image data representing a composite screen including at least two images based on at least two of the original data, the speckle data, the structure data and the mixed data.

6. An ultrasonic imaging apparatus according to claim 3, wherein said image data generating means generates image data representing a composite screen including an original image and a mixed image based on the original data and the mixed data.

7. An ultrasonic imaging apparatus according to claim 1, further comprising:
image processing means for performing image processing on at least one of the speckle data and the structure data.

8. An ultrasonic imaging apparatus according to claim 3, further comprising:
image processing means for performing image processing on at least one of the speckle data and the structure data.

9. An ultrasonic imaging apparatus according to claim 7, wherein said image processing means performs processing of dividing an ultrasonic image represented by one of the speckle data and the structure data into a plurality of frequency components, multiplying the plurality of frequency components by predetermined weighting factors respectively, and adding the plurality of frequency components multiplied by the weighting factors to each others.

10. An ultrasonic imaging apparatus according to claim 8, wherein said image processing means performs processing of dividing an ultrasonic image represented by one of the speckle data and the structure data into a plurality of frequency components, multiplying the plurality of frequency components by predetermined weighting factors respectively, and adding the plurality of frequency components multiplied by the weighting factors to each others.

11. An ultrasonic imaging apparatus according to claim 1, further comprising:
displaying means for displaying an image based on the image data generated by said image data generating means.

12. An ultrasonic imaging apparatus according to claim 3, further comprising:
displaying means for displaying an image based on the image data generated by said image data generating means.

13. An ultrasonic imaging apparatus according to claim 1, wherein said calculating means generates the structure data based on at least one of (i) local maximum points extracted from the original data, (ii) local minimum points extracted from the original data, (iii) average points of the local maximum points and the local minimum points, (iv) interpolation points having pixel values obtained by interpolation between the local maximum points, (v) interpolation points having pixel values obtained by interpolation between the local minimum points, and (vi) interpolation points having pixel values obtained by interpolation between the average points.

14. An ultrasonic imaging apparatus according to claim 3, wherein said calculating means generates the structure data based on at least one of (i) local maximum points extracted from the original data, (ii) local minimum points extracted from the original data, (iii) average points of the local maximum points and the local minimum points, (iv) interpolation points having pixel values obtained by interpolation between the local maximum points, (v) interpolation points having pixel values obtained by interpolation between the local minimum points, and (vi) interpolation points having pixel values obtained by interpolation between the average points.

15. An ultrasonic imaging apparatus according to claim 13, wherein said calculating means generates said structure data by performing two-dimensional mask processing.

16. An ultrasonic imaging apparatus according to claim 14, wherein said calculating means generates said structure data by performing two-dimensional mask processing.

17. An ultrasonic imaging apparatus according to claim 15, wherein said calculating means divides an ultrasonic image into four two-dimensional areas by using one of the interpolation points as a reference and performs four-point interpolation processing by using one of four local maximum points, four local minimum points and four average points of the four local maximum points and the four local minimum points selected in the respective areas to obtain a pixel value of the interpolation point.

18. An ultrasonic imaging apparatus according to claim 16, wherein said calculating means divides an ultrasonic image into four two-dimensional areas by using one of the interpolation points as a reference and performs four-point interpolation processing by using one of four local maximum points, four local minimum points and four average points of the four local maximum points and the four local minimum points selected in the respective areas to obtain a pixel value of the interpolation point.

19. An ultrasonic imaging apparatus according to claim 17, wherein said calculating means selects one of the four local maximum points, the four local minimum points and the four average points by using square interpolation masks.

20. An ultrasonic imaging apparatus according to claim 18, wherein said calculating means selects one of the four local maximum points, the four local minimum points and the four average points by using square interpolation masks.

21. An ultrasonic imaging apparatus according to claim 17, wherein said calculating means selects one of the four local maximum points, the four local minimum points and the four average points by using flat interpolation masks.

22. An ultrasonic imaging apparatus according to claim 18, wherein said calculating means selects one of the four local maximum points, the four local minimum points and the four average points by using flat interpolation masks.

23. An ultrasonic imaging apparatus according to claim 17, wherein said calculating means obtains a pixel value of the interpolation point by comparing pixel values based on said one of the four local maximum points, the four local minimum points and the four average points selected by using square interpolation masks and pixel values based on said one of the four local maximum points, the four local minimum points and the four average points selected by using flat interpolation masks.

24. An ultrasonic imaging apparatus according to claim 18, wherein said calculating means obtains a pixel value of the interpolation point by comparing pixel values based on said one of the four local maximum points, the four local minimum points and the four average points selected by using square interpolation masks and pixel values based on said one of the four local maximum points, the four local minimum points and the four average points selected by using flat interpolation masks.

25. An ultrasonic imaging apparatus according to claim 15, wherein said calculating means obtains a pixel value of the interpolation point based on pixel values of selected one of the local maximum points, the local minimum points and the average points extracted from the original data, and based on one of distances of the selected points and the interpolation point, squares of the distances and third powers of the distances.

26. An ultrasonic imaging apparatus according to claim 16, wherein said calculating means obtains a pixel value of the interpolation point based on pixel values of selected one of the local maximum points, the local minimum points and the average points extracted from the original data, and based on one of distances of the selected points and the interpolation point, squares of the distances and third powers of the distances.

27. An ultrasonic imaging apparatus according to claim 13, wherein said calculating means generates speckle data based on one of (i) differences between values represented by the original data and values represented by the structure data and (ii) values obtained by adding an offset value to the differences between values represented by the original data and values represented by the structure data.

28. An ultrasonic imaging apparatus according to claim 14, wherein said calculating means generates speckle data based on one of (i) differences between values represented by the original data and values represented by the structure data and (ii) values obtained by adding an offset value to the differences between values represented by the original data and values represented by the structure data.

29. An ultrasonic image processing method of generating an ultrasonic image based on original data representing ultrasonic image information on an object to be inspected and generated by transmitting ultrasonic waves and receiving ultrasonic echoes generated by reflection of the ultrasonic waves in the object so as to obtain detection signals and performing predetermined signal processing on the detection signals, said method comprising the steps of:
(a) generating speckle data representing image information on a speckle and structure data representing image information on a structure based on the original data; and
(b) generating image data based on at least one of the original data, the speckle data and the structure data.

30. An ultrasonic image processing method according to claim 29, further comprising the step of:
generating mixed data representing information on a mixed image in which a speckle image and a structure image are mixed with each other at a designated ratio based on the speckle data and the structure data;
wherein step (b) includes generating image data based on at least one of the original data, the speckle data, the structure data and the mixed data.

31. An ultrasonic image processing method of generating an ultrasonic image based on original data representing ultrasonic image information on an object to be inspected and generated by transmitting ultrasonic waves and receiving ultrasonic echoes generated by reflection of the ultrasonic waves in the object so as to obtain detection signals and performing predetermined signal processing on the detection signals, said method comprising the steps of:
(a) generating speckle data representing image information on a speckle and structure data representing image information on a structure based on the original data;
(b) generating mixed data representing information on a mixed image in which a speckle image and a structure image are mixed with each other at a designated ratio based on the speckle data and the structure data; and
(c) generating image data based on at least one of the original data and the mixed data.

32. An ultrasonic image processing method according to claim 29, further comprising the step of:
(d) performing image processing on at least one of the speckle data and the structure data.

33. An ultrasonic image processing method according to claim 31, further comprising the step of:
(d) performing image processing on at least one of the speckle data and the structure data.

34. An ultrasonic image processing method according to claim 32, wherein step (d) includes dividing an ultrasonic image represented by one of the speckle data and the structure data into a plurality of frequency components, multiplying the plurality of frequency components by predetermined weighting factors respectively, and adding the plurality of frequency components multiplied by the weighting factors to each others.

35. An ultrasonic image processing method according to claim 33, wherein step (d) includes dividing an ultrasonic image represented by one of the speckle data and the structure data into a plurality of frequency components, multiplying the plurality of frequency components by predetermined weighting factors respectively, and adding the plurality of frequency components multiplied by the weighting factors to each others.

36. An ultrasonic image processing method according to claim 29, wherein step (a) includes generating the structure data based on at least one of (i) local maximum points extracted from the original data, (ii) local minimum points extracted from the original data, (iii) average points of the local maximum points and the local minimum points, (iv) interpolation points having pixel values obtained by interpolation between the local maximum points, (v) interpolation points having pixel values obtained by interpolation between the local minimum points, and (vi) interpolation points having pixel values obtained by interpolation between the average points.

37. An ultrasonic image processing method according to claim 31, wherein step (a) includes generating the structure data based on at least one of (i) local maximum points extracted from the original data, (ii) local minimum points extracted from the original data, (iii) average points of the local maximum points and the local minimum points, (iv) interpolation points having pixel values obtained by interpolation between the local maximum points, (v) interpolation points having pixel values obtained by interpolation between the local minimum points, and (vi) interpolation points having pixel values obtained by interpolation between the average points.

38. An ultrasonic image processing method according to claim 36, wherein step (a) includes generating said structure data by performing two-dimensional mask processing.

39. An ultrasonic image processing method according to claim 37, wherein step (a) includes generating said structure data by performing two-dimensional mask processing.

40. An ultrasonic image processing program to be used for generating an ultrasonic image based on original data representing ultrasonic image information on an object to be inspected and generated by transmitting ultrasonic waves and receiving ultrasonic echoes generated by reflection of the ultrasonic waves in the object so as to obtain detection signals and performing predetermined signal processing on the detection signals, said program activating a CPU to execute the procedures of:
(a) generating speckle data representing image information on a speckle and structure data representing image information on a structure based on the original data; and
(b) generating image data based on at least one of the original data, the speckle data and the structure data.

41. An ultrasonic image processing program according to claim 40, further activating the CPU to execute the procedure of:
generating mixed data representing information on a mixed image in which a speckle image and a structure image are mixed with each other at a designated ratio based on the speckle data and the structure data;
wherein procedure (b) includes generating image data based on at least one of the original data, the speckle data, the structure data and the mixed data.

42. An ultrasonic image processing program to be used for generating an ultrasonic image based on original data representing ultrasonic image information on an object to be inspected and generated by transmitting ultrasonic waves and receiving ultrasonic echoes generated by reflection of the ultrasonic waves in the object so as to obtain detection signals and performing predetermined signal processing on the detection signals, said program activating a CPU to execute the procedures of:
(a) generating speckle data representing image information on a speckle and structure data representing image information on a structure based on the original data;
(b) generating mixed data representing information on a mixed image in which a speckle image and a structure image are mixed with each other at a designated ratio based on the speckle data and the structure data; and
(c) generating image data based on at least one of the original data and the mixed data.
